(19) United States
(12) Patent Application Publication
(10) Pub. No.: US 2025/0009880 A1
(43) Pub. Date: Jan. 9, 2025

(54) IMMUNE CELL THERAPY OF PD-L1 POSITIVE CANCERS

(71) Applicant: VITA THERAPEUTICS, INC., Baltimore, MD (US)

(72) Inventors: Aihong ZHANG, Baltimore, MD (US); Amir SABERI, Baltimore, MD (US); Douglas FALK, Baltimore, MD (US); Peter ANDERSEN, Baltimore, MD (US); Kayla Myers CHEN, Baltimore, MD (US)

(21) Appl. No.: 18/713,147

(22) PCT Filed: Nov. 28, 2022
(86) PCT No.: PCT/US2022/080504

§ 371 Date: May 23, 2024

Related U.S. Application Data
(60) Provisional application No. 63/283,387, filed on Nov. 26, 2021.

(51) Int. Cl. A61K 39/00; A61P 35/00

(52) U.S. Cl. CPC A61K 39/4631; A61K 39/4614; A61K 39/464411; A61P 35/00; A61K 2239/21; A61K 2239/22

(57) ABSTRACT

The present disclosure provides new anti-cancer immune cells engineered to express chimeric receptors which, unlike the conventional chimeric antigen receptors (CAR), employ the extracellular domain of PD-1 that is capable of binding PD-L1 that is expressed on a target tumor cell. The immune cell is preferably an immature myeloid cell that is p50 deficient. Such an engineered immune cell exhibits improved therapeutic efficacy as compared to the conventional immune cell therapies and is more broadly applicable to different types of cancers expressing, or induced to express PD-L1.

### CROSS REFERENCE TO RELATED APPLICATIONS

This application claims the benefit under 35 U.S.C. § 119 (e) of the U.S. Provisional Application Ser. No. 63/283,387, filed Nov. 26, 2021, the content of which is hereby incorporated by reference in its entirety.

### REFERENCE TO AN ELECTRONIC SEQUENCE LISTING

The contents of the electronic sequence listing (66CM-345112-WO.xml; Size: 25,541 bytes; and Date of Creation: Nov. 28, 2022) is herein incorporated by reference in its entirety.

### BACKGROUND

Checkpoint inhibitor therapies target immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD-1 on an immune cell surface, which inhibits immune cell activity. Among PD-L1 functions is a key regulatory role on T cell activities. Cancer-mediated upregulation of PD-L1 on the cell surface may inhibit T cells that might otherwise attack. Antibodies that bind to either PD-1 or PD-L1 and therefore block the interaction may allow the T-cells to attack the tumor.

Immune cell therapies, such as chimeric antigen receptor T cells (also known as CAR T cells), have been successfully developed to treat cancers. The efficacy of current immune cell therapies for treating solid cancers is limited by several factors. First, there is inherent heterogeneity among cancer cells. Second, myeloid-derived suppressor cells (MDSC) and/or tumor promoting M2 macrophages lead to a strong immunosuppressive tumor microenvironment (TME). Third, the conventional adoptive transferred immune cells are inefficient on tumor penetration.

Accordingly, there is a strong need for therapies that target these immune checkpoints and can penetrate the tumors to achieve higher efficacy.

### SUMMARY

The present disclosure provides new anti-cancer immune cell therapy approaches with improved therapeutic efficacy as compared to the conventional immune cell therapies. In particular, the present technology enables more effective penetration of solid tumors and is more broadly applicable to various different types of cancers that express PD-L1. It is contemplated that these added benefits are at least in part due to the new immune cell therapies' ability to modulate and reverse the immune suppressive TME, to enhance phagocytosis of tumor cells, and to increase activation of tumor-specific cytotoxic T cells.

In accordance with one embodiment of the present disclosure, therefore, provided is a method for treating a patient having a tumor cell that expresses programmed death ligand 1 (PD-L1), comprising administering to the patient an immune cell expressing a chimeric receptor comprising, from the N-terminus to the C-terminus, an extracellular domain of programmed cell death-1 (PD-1), a transmembrane domain, a costimulatory domain, and a CD3ξ intracellular domain. The tumor cell, in some embodiments, expresses PD-L1 on its own, or has been induced by a therapy to express PD-L1.

Example cancers that can be treated with the instantly disclosed method include triple negative breast cancer (TNBC), small cell lung cancer (SCLC), non-small lung cancer (NSCLC), melanoma, glioblastoma, prostate cancer, neuroblastoma, pancreatic ductal carcinoma, urothelial carcinoma, Merkel cell carcinoma, renal cell carcinoma (RCC), Hodgkin lymphoma (cHL), head and neck squamous cell cancer (HNSCC), gastric cancer, cervical cancer, microsatellite instability-high (MSI-H) or mismatch repair deficient (dMMR) metastatic colorectal cancer, and cutaneous squamous cell carcinoma (CSCC).

In some embodiments, the PD-1 extracellular domain comprises the amino acid sequence of SEQ ID NO:2 or 3. In some embodiments, the costimulatory domain is a signaling domain of a protein selected from the group consisting of CD28, CD27, OX40, CD40, CD80, CD86, and 4-1BB.

The immune cell used herein may be one of myeloid cell, natural killer (NK) cell, T cell, tumor infiltrating lymphocyte, and natural killer T (NKT) cell. In a preferred embodiment, the immune cell is an immature myeloid cell. In some embodiments, the immune cell is p50 deficient. In some embodiments, the immune cell does not express an active p50 or has reduced p50 activity.

In some embodiments, the immune cell further comprises an exogenous polynucleotide encoding a proinflammatory cytokine. Non-limiting examples include IL-12, IFN-γ, TNF-α, and IL-1β.

The therapeutic method, in some embodiments, is autologous, in which the immune cell is derived from a cell obtained from the patient. The derivation may include in vitro or ex vivo expansion. In some embodiments, the expansion is under a hypoxic condition.

### BRIEF DESCRIPTION OF THE DRAWINGS

FIG. 1A-B show the organization of Chimeric CAR-like Immune Receptor (CARIR) as well as an illustrative vector that encodes CARIR and other useful components of an engineered immune cell of the present technology. A. Illustration of CARIR, which comprises an extracellular domain (ED), a CD8 hinge region, a CD8 transmembrane domain, and CD3ξ activation domain. The ED is derived from natural human immune receptor, such as PD-1. B. Illustration of a lentiviral vector encoding CARIR and other useful components, including a protease digestion site P2A, a kill switch (truncated EGFR (tEGFR)), a protease digestion site T2A, and an IL-12 (including p40 and p35, connected through a short G₆S linker (SEQ ID NO:15) linker) as the additional proinflammatory cytokine. Upon protease treatments, the preprotein can be split into the chimeric receptor, the tEGFR kill switch, and the IL-12 cytokine. LTR: Long Terminal Repeats, EF1α: Elongation Factor 1α promoter, CARIR: CAR-Like Immune Receptor, P2A: Self-cleaving peptide sequences, tEGFR: Truncated Epidermal Growth Factor Receptor, PD-1: extracellular domain of PD-1 receptor, CD32: cytosolic domain of CD35, p40 & p35: subunits of interleukin IL-12.

FIG. 2A-B show lentiviral mediated CARIR and EGFP kill switch expressions in human monocytic THP-1 cells. THP-1 cells were transduced with lentiviral vector encoding PD-1 CARIR at indicated multiplicity of infection (MOI). A. Offset histograms show CARIR expression, measured by flow cytometry through detecting surface expression of PD-1. B. Offset histograms show the expression of truncated EGFR which serve as a safety kill switch. The cells were gated on live and singlets.

FIG. 3A-C show CARIR expression in transduced human monocytic THP-1 cells and its binding with human PD-L1 ligand. A. Diagram for a CARIR-z lentiviral vector that encoding a co-expressed Neon green marker. B. Co-expression of PD-1 and Neon green marker in transduced human monocytic THP-1 cells, as measured by flow cytometry. C. PD-1 CARIR expressed on transduced THP-1 cells binds biotinylated human PD-L1. CARIR transduced THP-1 cells were stained without or with increasing amount of biotinylated human PD-L1, followed by staining with streptavidin APC.

FIG. 4A-B show functional expression of CARIR in human monocytic THP-1 cells. A. Human PD-L1 expression in wild type RM-1 cells or RM-1^{hPD-L1}. The latter was engineered to over-express human PD-L1 through lentiviral mediated transduction. B. Upregulation of CD86 and to a much lesser degree, CD80, in CARIR transduced THP-1 cells after co-culture with RM-1 cells that engineered to express human PD-L1. THP-1 cells were either left not transduced (UTD) or transduced with human CARIR that lacks intracellular CD3 zeta chain (hCARIR-Δz), human CARIR (hCARIR-z), or a mouse analogue of hCARIR-z (mCARIR-z). The THP-1 cells were then stimulated with 1 ng/ml PMA for 24 hours, followed by the indicated co-culture treatment for 3 days. The cells were analyzed by flow cytometry for CD80 and CD86 expression. The cells were gated on THP-1 cells based on FSC/SSC parameter.

FIG. 5A-C show that CARIR expression in THP-1 macrophages potentiates pro-inflammatory cytokines production in response to LPS/IFN-γ stimulation. The effector THP-1 cells were pre-treated with 1 ng/ml PMA for 24 hours, and then stimulated with or without LPS+IFN-γ (20 ng/ml for each) for 3 days. The concentration of IL-6 (A), IL-1β (B), and TNF-α (C) cytokines in the culture supernatant was measured by ELISA assay. The experiment was performed in triplicate. The OD₄₅₀ₙₘ value shown was the absorbance (OD) at 450 nm wavelength after the OD value at the reference wavelength of 540 nm was subtracted. The data were expressed as mean±SD. N.d., not detectible. *p<0.05, ***p<0.001, ns: not significant, by one-way ANOVA using the Prism software. Multiplicity adjusted p values were reported.

FIG. 6A-F show that CD35 signaling domain is sufficient for CARIR functionality. For generating CARIR modified effector cells, human CD34+ hematopoietic stem cells (HSCs) were engineered to express CARIR through lentiviral transduction, and then differentiated into macrophages (MP). For target cells, both wild type RM-1 cells and RM-1^{hPD-L1} cells were used. The later line was established by overexpressing human PD-L1 through lentiviral vector-mediated transduction. A. A flow chart for the experimental procedures. B. The flow data showing the % phagocytosis of human CARIR-z MΦ on wild type RM-1 or RM-1^{hPD-L1} target cells. C. The flow data showing the % phagocytosis of human CARIR-40z MP on wild type RM-1 or RM-1^{hPD-L1} target cells. D. The flow data showing the % phagocytosis of human CARIR-40 MQ on wild type RM-1 or RM-1^{hPD-L1} target cells. E. The flow data showing the % phagocytosis of human CARIR-z-12 MΦ on wild type RM-1 or RM-1^{hPD-L1} target cells. F. The bar graph summarizes the flow data shown in panels from B to E. The cells were gated on CellTrace Violet⁺ and PD-1⁺ MΦ. The CellTrace Violet and CFSE double positive population represent MΦ that have phagocytosed target cells. **p<0.01, ***p<0.001, and ****p<0.0001 by unpaired student t test analyzed using Prism software.

FIG. 7A-C show CARIR expression in human monocytic THP-1 cells increases phagocytosis on RM-1^{hPD-L1} target cells. Phagocytosis assay was utilized to evaluate the functionality of CARIR. The CellTrace Violet labeled effector THP-1, CARIR-Δz THP-1 (lacking CD33 signaling domain in the CARIR), or CARIR-z THP-1 cells were pretreated with 1 ng/ml PMA for 24 hours. The effector cells were then co-cultured for 4 hours with CFSE labeled RM-1 or RM-1^{hPD-L1} (RM-1 cells that engineered to overexpress human PD-L1) target cells at the effector to target ratio of 5:1, in the presence or absence of 2 μM cytochalasin D (cyto), 10 μg/ml pembrolizumab biosimilar anti-PD-1 antibody (aPD1), or human IgG4 isotype control (iso). The % phagocytosis was assessed by flow cytometry. A. CARIR transduction efficiency in THP-1 cells as evaluated by flow staining for PD-1. The cells were gated on live, singlets. B. Example flow dot plots showing the % phagocytosis by CARIR-z-THP-1 cells in the presence or absence of RM-1^{hPD-L1} target cells. The cells were gated on live, singlets, and Violet⁺CFSE⁺. C. The bar graph summarizes the % phagocytosis result. The data were expressed as mean±SD. *p<0.05, ****p<0.0001, and ns: not significant, by one-way ANOVA using the Prism software. Multiplicity adjusted p values were reported.

FIG. 8 shows Efficient NFκB-1 (p50) gene knockout in human HSCs through CRISPR/Cas9 approach. Human mobilized peripheral blood (MPB) or bone marrow (BM)-derived CD34⁺ hematopoietic stem cells (HSCs) were electroporated with ribonucleoprotein (RNP) complex containing recombinant Cas9 protein as well as guide RNA #1 (gRNA #1, target: TACCCGACCACCATGTCCTT; SEQ ID NO:20) and/or guide RNA #2 (gRNA #2, target: ATATAGATCTGCAACTATGT; SEQ ID NO:21). The % indel among the NFκB-1 (p50) gene was measured by TIDE analysis 6 days later.

FIG. 9A-B show the Engineering of primary human CD34+ hematopoietic stem cells (HSCs) to knock out NF-kB1 (p50) and express CARIR. A. Procedure for the same-day human HSCs engineering. B. Offset histograms show CARIR (PD-1) expression on HSCs with or without NF-kB1 (p50) knock out. The cells were gated on live and singlets. NT: no transduction; Mock: mock transduction; CARIR: CARIR transduction only; p50 KO: p50 knock-out only.

FIG. 10A-B show the in vitro expansion of human CD34⁺ hematopoietic stem cells (HSCs). Mobilized peripheral blood (MPB) or bone marrow (BM)-derived human CD34⁺ HSCs were expanded in vitro for a week in culture medium containing TPO, SCF, Flt3L, and UM171. The number of live cells were counted at the indicated time points. A. Fold expansion of MPB-derived CD34⁺ HSCs. The expansion experiment was conducted with cells from 2 different donors, each with 3 independent repeats. The data was shown as mean±SEM. B. Fold expansion of BM-derived CD34⁺ HSCs. The expansion experiment was conducted with cells from one donor in duplicate. Average number of the cells were shown in the figure at each timepoint.

FIG. 11A-D show increased myeloid differentiation of BM versus MPB-derived human CD34⁺ HSCs under either normoxia or hypoxia conditions. Mobilized peripheral blood (MPB) or bone marrow (BM)-derived human CD34⁺ HSCs were plated in ultralow attachment plates in myeloid differentiation medium containing M-CSF and GM-CSF. The cells were cultured in the incubator that either maintain normoxia (20% O₂) or hypoxia (1% O₂) as indicated. On day 4, 7, and 10, the cells were analyzed by flow cytometry for cell surface markers CD11b and CD34. A. The percentage of CD11b⁺ myeloid cells differentiated under normoxia condition over the time course. B. The percentage of cells that remain to be CD34⁺ following myeloid differentiated under normoxia condition over the time course. C. The percentage of CD11b⁺ myeloid cells differentiated under hypoxia condition over the time course. D. The percentage of cells that remain to be CD34⁺ following myeloid differentiated under hypoxia condition over the time course. The experiment was conducted in triplicate and 2 to 3 different donors of each sample types were used. Data was presented as mean±SEM. *p<0.05 comparing the BM versus MPB cell sample by student t test with 2-tailed distribution.

FIG. 12A-D show that CARIR expression in human monocytic THP-1 cells increases their phagocytosis on PD-L1⁺ human triple negative breast cancer cells. To serve as the effectors, human monocytic THP-1 cells were engineered to express either CARIR-Δz (a PD-1 CARIR that lacks CD3ζ signaling domain) or CARIR-z through lentiviral transduction, and then differentiated to macrophages by PMA treatment. MDA-MB-231 tumor cells that express PD-L1 were used to serve as the target cells. A. A flow chart for the experimental procedures. B. Histogram shows positive PD-L1 expression on MDA-MB-231 cells. C. Representative flow plots showing % phagocytosis on MDA-MB-231 tumor cells. The cells were gated on CellTrace Violet THP-1 macrophages. The CellTrace Violet and CellTrace Yellow double positive population represent macrophages that have phagocytosed target cells. D. Bar graph summarizes the phagocytosis result shown in panel C. ***p<0.001, and ****p<0.0001 by ordinary one-way ANOVA using Prism software.

FIG. 13A-B show that CARIR expression increases the phagocytosis of human THP-1 macrophages on PD-L1⁺ NCI-H358 human lung cancer cells. A. Histogram shows high level of PD-L1 expression in the cultured NCI-H358 tumor cells. B. % phagocytosis on NCI-H358 cells. Human monocytic THP-1 cells were engineered to express either CARIR-Δz or CARIR-z through lentiviral transduction, and then differentiated to macrophages by PMA pretreatment. CellTrace Violet labeled THP-1 effectors were co-cultured with CellTrace Yellow labeled NCI-H358 tumor cells for 4 hours, followed by flow cytometry analysis of the % phagocytosis. Cells were gated on live, singlets, and violet⁺ cells. The events that were double positive for CellTrace Violet and CellTrace Yellow were considered as phagocytic events. Data was presented as mean±SD. *p<0.05, and **p<0.01 by ordinary one-way ANOVA using Prism software.

FIG. 14A-B show that CARIR expression increases the phagocytosis of human THP-1 macrophages on PD-L1+BT-549 human triple negative breast cancer cells. A. Histogram shows high level of PD-L1 expression in the cultured BT-549 tumor cells. B. % phagocytosis on BT-549 cells. Human monocytic THP-1 cells were engineered to express either CARIR-Δz or CARIR-z through lentiviral transduction, and then differentiated to macrophages by PMA pretreatment. CellTrace Violet labeled THP-1 effectors were co-cultured with CellTrace Yellow labeled BT-549 tumor cells for 4 hours, followed by flow cytometry analysis of the % phagocytosis. Cells were gated on live, singlets, and violet⁺ cells. The events that were double positive for CellTrace Violet and CellTrace Yellow were considered as phagocytic events. Data was presented as mean±SD. **p<0.01, and ****p<0.0001 by ordinary one-way ANOVA using Prism software.

FIG. 15A-B show that there was no increased phagocytosis by CARIR THP-1 macrophages on PD-L1 low or negative human tumor cells. A. Histogram shows the expression levels of PD-L1 on the indicated cultured tumor cells. B. Summary bar graph shows the % phagocytosis on each type of the tumor cells. Human monocytic THP-1 cells were engineered to express either CARIR-Δz or CARIR-z through lentiviral transduction, and then differentiated to macrophages by PMA pretreatment. CellTrace Violet labeled THP-1 effectors were co-cultured with CellTrace Yellow labeled indicated tumor cells for 4 hours, followed by flow cytometry analysis of the % phagocytosis. Cells were gated on live, singlets, and violet+ cells. The events that were double positive for CellTrace Violet and CellTrace Yellow are considered as phagocytic events. The experiment was conducted in triplicate. No increase of phagocytosis was observed for the indicated PD-L1 low or negative human tumor cells.

FIG. 16A-D show that Infusion of engineered myeloid cells slows 4T1 tumor growth and prolongs survival in syngeneic mouse model. A. Schematic timeline for the animal experiment. Syngeneic Balb/c mice were subcutaneously implanted with 5×10⁴ 4T1 breast cancer cells on day −7. Starting on day 0, the mice were treated with 3 weekly dose of 10×10⁶ engineered mouse immature myeloid cells expressing murine analog of CARIR (CARIR-IMC) or with NFκB-1 (p50) knocked out (p50^{-/-} IMC) or PBS as indicated. B. Tumor volume measurements. C. Probability of survival. D. Body weight over the course of the experiment. Data are presented as mean±SEM. *p<0.01: CARIR-IMC vs WT-IMC or PBS, p50^{-/-} IMC vs WT-IMC or PBS, One-way ANOVA. #p<0.05: CARIR-IMC vs PBS, p<0.01: CARIR-IMC vs WT-IMC, p50^{-/-} IMC vs WT-IMC or PBS, by Gehan-Breslow-Wilcoxon test using prism software.

FIG. 17 illustrates the process of the immune cell therapy for patient with PD-L1+ cancer.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

### PD-L1 Targeting Chimeric Receptors

In various aspects of the present technology, an immune cell is transduced to express a chimeric receptor that helps target the immune cell to a tumor cell. The chimeric receptor, also referred to as a “CAR-like immune receptor,” or “CARIR”, like a conventional CAR, includes an extracellular targeting domain, a transmembrane domain, and one or more costimulatory domains or signal domains.

A conventional CAR includes an antibody or antigen-binding fragment, such as a single chain fragment (scFv), as the extracellular targeting domain to bind to a target molecule, such as a tumor-associated antigen (TAA). By contrast, the chimeric receptors of the present disclosure, in some embodiments, employs the extracellular binding domain of a natural receptor protein that can bind to the target protein, through a conventional ligand-receptor interaction.

For instance, various inhibitory receptors are expressed on immune cells, such as myeloid cells. Non-limiting examples include PD-1 which can bind to ligand PD-L1, SIRPα which can bind to CD47, Siglec-10 which can bind to CD52 and CD24, CTLA-4 which can bind to B7-1 and B7-2, TIM-3 which can bind to Gal-9, PtdSer, HMGB1 and CEACAM1, and LAG3 which can bind to MHC class II and FGL1. In a particular embodiment, the receptor protein is PD-1.

As demonstrated in the accompanying experimental examples, when a CARIR that contained the extracellular domain of PD-1 was expressed on an immune cell, such as an immature myeloid cell or a macrophage, the engineered immune cell was able to bind to tumor cells expressing PD-L1 and initiate phagocytosis (Examples 3 and 6), and adoptive transfer CARIR-expressing immature myeloid cells leading to inhibition of tumor growth (Example 7).

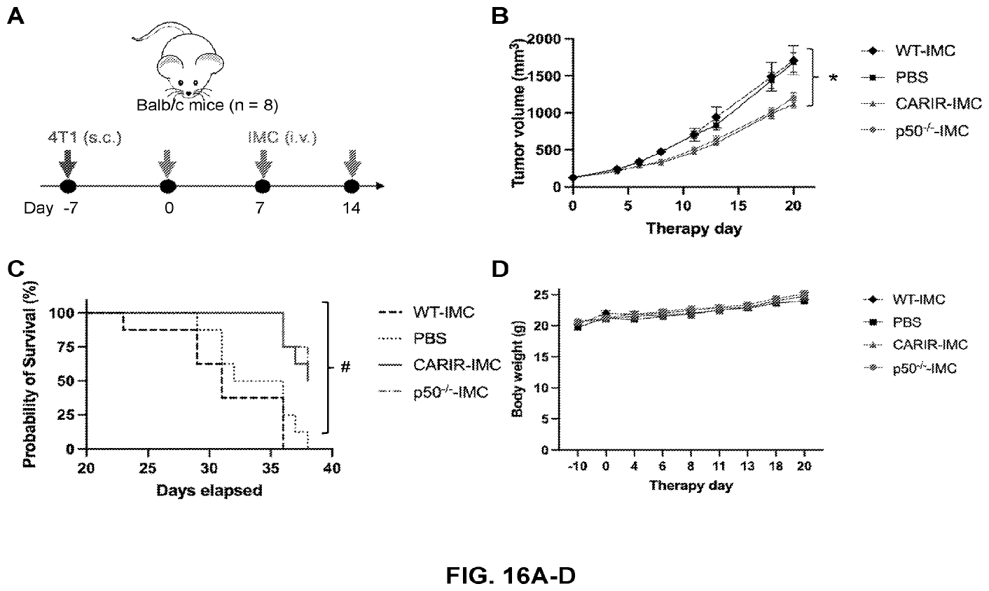

Such anti-tumor effects of the CARIR molecules, however, were unexpected. It is commonly known that therapeutic antibodies typically have a binding affinity on the scale of 0.1-10 nM (EC₅₀). For instance, the EC₅₀ of anti-PD1 antibodies pembrolizumab and nivolumab are 2.440 nM and 5.697 nM, respectively. The affinity between the natural ligands and receptors, however, can be considerably lower. For example, the EC₅₀ between PD-1 and PD-L1 is 7 UM and that between SIRPα and CD47 is 2 μM, both of which are about 1000 times weaker than antibodies. With a 1000-fold lower binding affinity but achieving in vivo anti-tumor effects in a model which is known for its poor response to traditional anti-PD1/PD-L1 antibody blockade therapy (FIG. 16B-C), the instantly disclosed CARIR truly have exhibited unexpected results.

The full-length sequence of PD-1 is provided in Table 1, along with its extracellular targeting domains.

TABLE 1
Extracellular Domains of PD-1
Sequences

Full length: (SEQ ID NO: 1)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL
Core ECD binding sequence: (SEQ ID NO: 2)
MSPSNQTDKLAAFPEDRSQPGQDCRFRVTQ
Extended ECD binding sequence: (SEQ ID NO: 3)
MSPSNQTDKLAAFPEDRSQPGQDCRFRVTQLPNGRDFHMSVVRARRNDS
GTYLCGAISLAPKAQIKESLRAELRVTERRAEVPTAHPSPSPRPAGQFQ
TLV

Accordingly, in some embodiments, a chimeric receptor is provided which includes, from the N-terminus to the C-terminus, an extracellular domain of PD-1, a transmembrane domain, a costimulatory domain, and a CD3ξ intracellular domain. In some embodiments, the extracellular domain includes a ligand-binding domain.

The extracellular targeting domain can target the engineered immune cell, which expresses the extracellular targeting domain of PD-1, to a tumor tissue where PD-L1 is expressed. In addition to the extracellular domain, the chimeric receptor also includes other useful elements.

In some embodiments, the chimeric receptor further includes a transmembrane (TM) domain. A transmembrane domain can be designed to be fused to the extracellular domain, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-IBB employed as a costimulatory domain) or the natural transmembrane domain of a hinge region (e.g., the TM region of a CD8 alpha or CD28T employed as a hinge domain). Example sequences are provided in Table 2.

In some embodiments, the transmembrane domain can be selected or modified by amino acid substitution to avoid binding of such domains to the transmembrane domains of the same or different surface membrane proteins to minimize interactions with other members of the receptor complex. A transmembrane domain can be derived either from a natural or from a synthetic source. When the transmembrane domain is derived from a naturally-occurring source, the domain can be derived from any membrane-bound or transmembrane protein. In some embodiments, a transmembrane domain is derived from CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB 1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF 1), CD158A (KTR2DL1), CD158B1 (KIR2DL2), CD158B2 (KIR2DL3), CD158C (KTR3DP1), CD158D (KTRDL4), CD158F 1 (KTR2DL5A), CD158F2 (KTR2DL5B), CD158K (KIR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (T FRSF18), inducible T cell co-stimulator (ICOS), LFA-1 (CD11a/CD18), KG2C, DAP-10, ICAM-1, Kp80 (KLRF 1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and combinations thereof.

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain.”

In some embodiments, the transmembrane domain of a chimeric receptor of the instant disclosure includes the human CD8α transmembrane domain (SEQ ID NO:7). In some embodiments, the CD8a transmembrane domain is fused to the extracellular domain through a hinge region. In some embodiments, the hinge region includes the human CD8a hinge (SEQ ID NO:6).

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the chimeric receptor further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CDIld (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (TFSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD11a/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the costimulatory domain is selected from the group consisting of CD80, CD86, CD40, 41BB, OX40, and CD28. Some example sequences are provided is Table 2.

In some embodiments, the cytoplasmic portion of the chimeric receptor also includes a signaling/activation domain. In one embodiment, the signaling/activation domain is the CD3ξ domain (SEQ ID NO:18), or is an amino acid sequence having at least about 80%, 85%, 90%, 95%, 98% or 99% sequence identity to the CD3ξ domain.

In some embodiments, the chimeric receptor also includes a leader peptide (also referred to herein as a “signal peptide” or “signal sequence”). The inclusion of a signal sequence in a chimeric receptor is optional. If a leader sequence is included, it can be expressed on the N terminus of the chimeric receptor. Such a leader sequence can be synthesized, or it can be derived from a naturally occurring molecule. An example leader peptide is the human CSF-2 signal peptide (SEQ ID NO:5).

In some embodiments, the chimeric receptor of the present disclosure includes a leader peptide (P), an extracellular targeting domain (T), a hinge domain (H), a transmembrane domain (T), one or more costimulatory regions (C), and an activation domain (A), wherein the chimeric receptor is configured according to the following: P-T-H-T-C-A. In some embodiments the components of the chimeric receptor are optionally joined though a linker sequence, such as AAA or GSG. Some example sequences are provided in Table 2.

TABLE 2
Representative Elements of the Chimeric Receptor

| Elements | Sequences |
|---|---|
| human EF1α | GGCTCCGGTGCCCGTCAGTGGGCAGAGCGCACATCGCCCACAGTCCCCGAGAAGTTGG |
| promoter (SEQ | GGGGAGGGGTCGGCAATTGAACCGGTGCCTAGAGAAGGTGGCGCGGGGTAAACTGGGA |
| ID NO: 4) | AAGTGATGTCGTGTACTGGCTCCGCCTTTTTCCCGAGGGTGGGGGAGAACCGTATATA |
| | AGTGCAGTAGTCGCCGTGAACGTTCTTTTTCGCAACGGGTTTGCCGCCAGAACACAGG |
| | TAAGTGCCGTGTGTGGTTCCCGCGGGCCTGGCCTCTTTACGGGTTATGGCCCTTGCGT |
| | GCCTTGAATTACTTCCACCTGGCTGCAGTACGTGATTCTTGATCCCGAGCTTCGGGTT |
| | GGAAGTGGGTGGGAGAGTTCGAGGCCTTGCGCTTAAGGAGCCCCTTCGCCTCGTGCTT |
| | GAGTTGAGGCCTGGCCTGGGCGCTGGGGCCGCCGCGTGCGAATCTGGTGGCACCTTCG |
| | CGCCTGTCTCGCTGCTTTCGATAAGTCTCTAGCCATTTAAAATTTTTGATGACCTGCT |
| | GCGACGCTTTTTTTCTGGCAAGATAGTCTTGTAAATGCGGGCCAAGATCTGCACACTG |
| | GTATTTCGGTTTTTGGGGCCGCGGGCGGCGACGGGGCCCGTGCGTCCCAGCGCACATG |
| | TTCGGCGAGGCGGGGCCTGCGAGCGCGGCCACCGAGAATCGGACGGGGGTAGTCTCAA |
| | GCTGGCCGGCCTGCTCTGGTGCCTGGTCTCGCGCCGCCGTGTATCGCCCCGCCCTGGG |
| | CGGCAAGGCTGGCCCGGTCGGCACCAGTTGCGTGAGCGGAAAGATGGCCGCTTCCCGG |
| | CCCTGCTGCAGGGAGCTCAAAATGGAGGACGCGGCGCTCGGGAGAGCGGGCGGGTGAG |
| | TCACCCACACAAAGGAAAAGGGCCTTTCCGTCCTCAGCCGTCGCTTCATGTGACTCCA |
| | CGGAGTACCGGGCGCCGTCCAGGCACCTCGATTAGTTCTCGAGCTTTTGGAGTACGTC |
| | GTCTTTAGGTTGGGGGGAGGGGTTTTATGCGATGGAGTTTCCCCACACTGAGTGGGTG |
| | GAGACTGAAGTTAGGCCAGCTTGGCACTTGATGTAATTCTCCTTGGAATTTGCCCTTT |
| | TTGAGTTTGGATCTTGGTTCATTCTCAAGCCTCAGACAGTGGTTCAAAGTTTTTTTCT |
| | TCCATTTCAGGTGTCGTGA |
| human CSF-2 | MWLQSLLLLGTVACSIS |
| signal peptide | |
| (SEQ ID NO: 5) | |
| human CD8 | KPTTTPAPRPPTPAPTIASQPLSLRPEACRPAAGGAVHTRGLDFACDIY |
| hinge (SEQ ID | |
| NO: 6) | |
| human CD8 | IWAPLAGTCGVLLLSLVITLY |
| transmembrane | |
| domain (SEQ ID | |
| NO: 7) | |
| P2A protease | ATNFSLLKQAGDVEENPGP |
| site (SEQ ID | |
| NO: 8) | |
| T2A protease | EGRGSLLTCGDVEENPGP |
| site (SEQ ID | |
| NO: 9) | |
| human CD40 | KKVAKKPTNKAPHPKQEPQEINFPDDLPGSNTAAPVQETLHGCQPVTQEDGKESRISV |
| isoform 1 | QERQ |
| cytoplasmic | |
| domain (SEQ ID | |
| NO: 10) | |
| human IL-12 | Coding sequence (SEQ ID NO: 11) |
| fusion (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGTGG |
| | AGGTGGCGGCGGCGGCTCTCGAAATCTGCCAGTCGCTACCCCGGACCCAGGAATGTTT |
| | CCATGCCTGCACCACAGTCAGAACCTGCTCCGGGCGGTTTCCAACATGCTTCAGAAAG |
| | CGCGCCAGACCCTTGAATTTTACCCCTGCACAAGTGAAGAGATAGACCATGAAGATAT |
| | TACCAAGGATAAAACATCAACTGTAGAGGCGTGTCTCCCTCTCGAACTGACAAAGAAC |
| | GAGTCTTGTCTCAATAGTAGGGAAACTTCATTCATTACAAACGGGTCATGTCTTGCTT |
| | CAAGGAAGACCAGCTTCATGATGGCACTCTGCTTGTCTTCAATCTATGAGGATCTTAA |
| | AATGTACCAAGTAGAGTTTAAGACTATGAATGCGAAGCTCCTGATGGATCCGAAGCGG |
| | CAGATTTTTTTGGACCAGAATATGTTGGCGGTCATTGACGAACTTATGCAAGCTCTCA |
| | ATTTCAATTCAGAGACGGTTCCTCAGAAAAGCTCCTTGGAAGAGCCGGACTTCTACAA |
| | AACTAAGATCAAATTGTGTATCTTGCTCCATGCATTCCGGATACGCGCCGTGACCATT |
| | GATCGAGTAATGTCCTATTTGAATGCAAGCTAA |
| | Amino acid sequence (SEQ ID NO: 12) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVF |
| | TDKTSATVICRKNASISVRAQDRYYSSSWSEWASVPCSGGGGGGSRNLPVATPDPGMF |
| | PCLHHSQNLLRAVSNMLQKARQTLEFYPCTSEEIDHEDITKDKTSTVEACLPLELTKN |
| | ESCLNSRETSFITNGSCLASRKTSFMMALCLSSIYEDLKMYQVEFKTMNAKLLMDPKR |
| | QIFLDQNMLAVIDELMQALNFNSETVPQKSSLEEPDFYKTKIKLCILLHAFRIRAVTI |
| | DRVMSYLNAS |
| human IL-12B/ | Coding sequence (SEQ ID NO: 13) |
| p40 (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGT |
| | Amino acid sequence (SEQ ID NO: 14) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVF |
| | TDKTSATVICRKNASISVRAQDRYYSSSWSEWASVPCS |
| G6S linker (SEQ | GGGGGGS |
| ID NO: 15) | |
| human IL-12A/ | Coding sequence (SEQ ID NO: 16) |
| p35 (codon | CGAAATCTGCCAGTCGCTACCCCGGACCCAGGAATGTTTCCATGCCTGCACCACAGTC |
| optimized) | AGAACCTGCTCCGGGCGGTTTCCAACATGCTTCAGAAAGCGCGCCAGACCCTTGAATT |
| | TTACCCCTGCACAAGTGAAGAGATAGACCATGAAGATATTACCAAGGATAAAACATCA |
| | ACTGTAGAGGCGTGTCTCCCTCTCGAACTGACAAAGAACGAGTCTTGTCTCAATAGTA |
| | GGGAAACTTCATTCATTACAAACGGGTCATGTCTTGCTTCAAGGAAGACCAGCTTCAT |
| | GATGGCACTCTGCTTGTCTTCAATCTATGAGGATCTTAAAATGTACCAAGTAGAGTTT |
| | AAGACTATGAATGCGAAGCTCCTGATGGATCCGAAGCGGCAGATTTTTTTGGACCAGA |
| | ATATGTTGGCGGTCATTGACGAACTTATGCAAGCTCTCAATTTCAATTCAGAGACGGT |
| | TCCTCAGAAAAGCTCCTTGGAAGAGCCGGACTTCTACAAAACTAAGATCAAATTGTGT |
| | ATCTTGCTCCATGCATTCCGGATACGCGCCGTGACCATTGATCGAGTAATGTCCTATT |
| | TGAATGCAAGCTAA |
| | Amino acid sequence (SEQ ID NO: 17) |
| | RNLPVATPDPGMFPCLHHSQNLLRAVSNMLQKARQTLEFYPCTSEEIDHEDITKDKTS |
| | TVEACLPLELTKNESCLNSRETSFITNGSCLASRKTSFMMALCLSSIYEDLKMYQVEF |
| | KTMNAKLLMDPKRQIFLDQNMLAVIDELMQALNENSETVPQKSSLEEPDFYKTKIKLC |
| | ILLHAFRIRAVTIDRVMSYLNAS |
| CD3ζ domain | RVKFSRSADAPAYQQGQNQLYNELNLGRREEYDVLDKRRGRDPEMGGKPRRKNPQEGL |
| (SEQ ID NO: 18) | YNELQKDKMAEAYSEIGMKGERRRGKGHDGLYQGLSTATKDTYDALHMQALPPR |
| Truncated EGFR | RKVCNGIGIGEFKDSLSINATNIKHFKNCTSISGDLHILPVAFRGDSFTHTPPLDPQE |
| (SEQ ID NO: 19) | LDILKTVKEITGFLLIQAWPENRTDLHAFENLEIIRGRTKQHGQFSLAVVSLNITSLG |
| | LRSLKEISDGDVIISGNKNLCYANTINWKKLFGTSGQKTKIISNRGENSCKATGQVCH |
| | ALCSPEGCWGPEPRDCVSCRNVSRGRECVDKCNLLEGEPREFVENSECIQCHPECLPQ |
| | AMNITCTGRGPDNCIQCAHYIDGPHCVKTCPAGVMGENNTLVWKYADAGHVCHLCHPN |
| | CTYGCTGPGLEGCPTNGPKIPSIATGMVGALLLLLVVALGIGLFM |

### Engineered Immune Cells

The chimeric receptors disclosed herein can be expressed in an immune cell which can be suitably used for therapeutic purposes. Example immune cells include myeloid cells, natural killer (NK) cells, T cells, tumor infiltrating lymphocytes, and natural killer T (NKT) cells. The preparation and use of T cells transduced to express chimeric antigen receptors (CAR) have been well described in the art. The instantly disclosed chimeric receptors can likewise be expressed in T cells, and are used like CAR-T cells. Nevertheless, the present technology is not limited to T cells.

In some embodiments, the immune cell is a myeloid cell, in particular an immature myeloid cell (IMC).

Myeloid cells are produced by hematopoietic stem cells. Myeloid cells are progenitor cells which can produce different types of blood cells including monocytes, macrophages, neutrophils, basophils, eosinophils, erythrocytes, dendritic cells, megakaryocytes, and platelets. Myeloid cells originate in bone marrows. Myeloid cells encompass circulating progenitor monocytes and tissue resident macrophage cells, including hepatic Kupffer cells, lymph-associated macrophages in spleen and lymph nodes, Langerhans cells in the skin, pulmonary alveolar macrophages, and highly specialized dendritic cells found primarily along mucosal surfaces.

“Immature myeloid cells” (IMC), “early myeloid cells,” “myeloid suppressive cells,” or “myeloid-derived suppressor cells” (MDSCs), are progenitor cells present in the bone marrow and spleen of healthy subjects which can differentiate into mature myeloid cells under normal conditions. These cells are associated with immune suppression during viral infection, transplantation, UV irradiation and cyclophosphamide (CTX) treatment. It has also been shown that the accumulation of IMC within the tumor microenvironment correlates with a poor prognosis. The instant inventors have the insight that immunotherapy with such cells can promote penetration into the tumor microenvironment.

The IMC can be prepared using established methods from selected autologous cell sources, such as CD34+ hematopoietic stem cells from the bone marrow or mobilized CD34+ hematopoietic stem cells from the peripheral blood. Alternatively, the IMC can be generated in vitro from induced pluripotent stem cells (iPSCs).

In some embodiments, the progenitor cells or immune cells can be cultured or expanded in a medium under a hypoxic condition. In some embodiments, the hypoxic condition is induced by a cobalt salt in the medium, such as 20 μM to 200 μM CoCl₂, or preferably 50 μM to 150 μM CoCl₂, in the medium. Alternatively, in some embodiments, the hypoxic condition is induced by placing the medium in a chamber having no more than 10% oxygen in the air, preferably no more than 5%, or 2% or 1% oxygen in the air.

Collection of CD34⁺ cells from adult bone marrow can be challenging and the amount collected is typically low. An alternative approach is to use mobilized peripheral blood (MPB). MPB is collected from healthy donors that are injected with Granulocyte-Colony Stimulating Factor (G-CSF), Plerixafor, or a combination of Plerixafor and G-CSF. These mobilization agents increase circulating leukocytes and stimulate the bone marrow to produce a large number of hematopoietic stem cells, which are mobilized into the bloodstream, allowing for large quantities of stem cells and MNCs to be collected from a single donor.

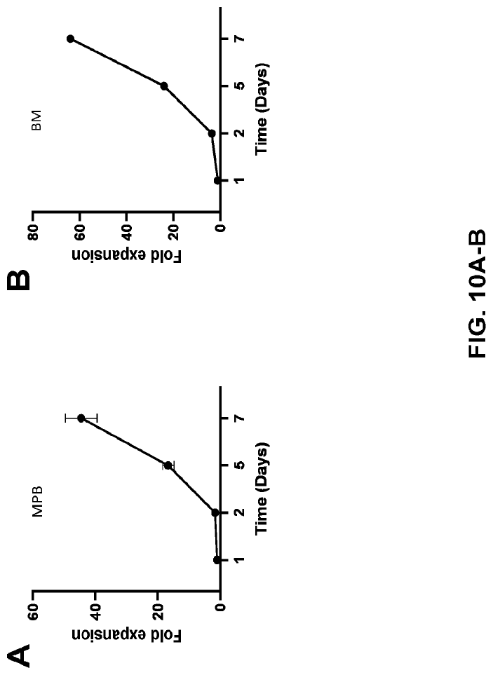

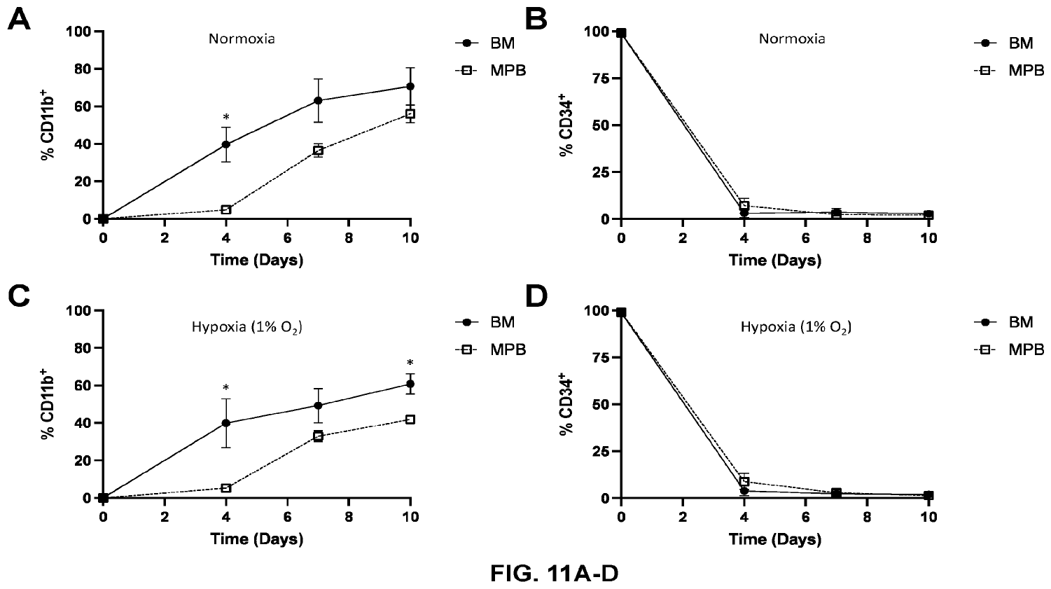

In a surprising discovery, however, the instant inventors observed that more myeloid cells can be generated from differentiation of bone marrow-derived human CD34⁺ cells than from MPB-derived human CD34⁺ cells, under both normoxia and hypoxia conditions (FIGS. 10 and 11). In some embodiments, therefore, the bone marrow is the preferred source of progenitor cells.

In some embodiments, the immune cell is engineered to be p50 deficient. NF-κB p50 (nuclear factor NF-kappa-B p105 subunit) is a Rel protein-specific transcription inhibitor, and is the DNA binding subunit of the NF-kappaB (NF-κB) protein complex. NF-κB is a transcription factor that is activated by various intra- and extra-cellular stimuli such as cytokines, oxidant-free radicals, ultraviolet irradiation, and bacterial or viral products. Activated NF-κB translocates into the nucleus and stimulates the expression of genes involved in a wide variety of biological functions. p50 is an inhibitory subunit; in the basal state p65 is held in the cytoplasm by IκB, whereas p50:p50 homo-dimers enter the nucleus, bind DNA, and repress gene expression. Absence of p50 leads to activation of pro-inflammatory pathways.

In some embodiments, a p50 deficient immune cell is an immune cell that has been engineered to have reduced expression or biological activity of the p50 gene. In some embodiments, a p50 deficient immune cell is an immune cell in which the p50 gene is knocked out (p50^{-/-}). Reduced expression or biological activity or knock-out can be readily implemented with techniques well known in the art, such as CRISPR. In some embodiments, a single allele of the p50 gene is inactivated; in some embodiments, both alleles of the p50 gene are inactivated. In some embodiments, the immune cell is a p50^{-/-} immature myeloid cell.

In some embodiments, the immune cell is further engineered to produce a proinflammatory cytokine. Example proinflammatory cytokines include the IL-1 family (e.g., IL18, IL18BP, IL1A, IL1B, IL1F10, IL1F3/IL1RA, IL1F5, IL1F6, IL1F7, IL1F8, IL1RL2, IL1F9, and IL33), IL-1 receptors (e.g., IL18R1, IL18RAP, IL1R1, IL1R2, IL1R3, IL1R8, IL1R9, IL1RL1, and SIG1RR), the TNF family (BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, and TNFSF4), TNF receptors (e.g., 4-1BB, BAFFR, TNFRSF7, CD40, CD95, DcR3, TNFRSF21, EDA2R, EDAR, PGLYRP1, TNFRSF19L, TNFR1, TNFR2, TNFRSF11A, TNFRSF11B, TNFRSF12A, TNFRSF13B, TNFRSF14, TNFRSF17, TNFRSF18, TNFRSF19, TNFRSF25, LTBR, TNFRSF4, TNFRSF8, TRAILR1, TRAILR2, TRAILR3, and TRAILR4), Interferons (IFN) (e.g., IFNA1, IFNA10, IFNA13, IFNA14, IFNA2, IFNA4, IFNA7, IFNB1, IFNE, IFNG, IFNZ, IFNA8, IFNA5/IFNaG, and IFNω/IFNW1), IFN receptors (e.g., IFNAR1, IFNAR2, IFNGR1, and IFNGR2), the IL6 family (e.g., CLCFI, CNTF, IL11, IL31, IL6, Leptin, LIF, OSM, IL6 Receptor, CNTFR, IL11RA, IL6R, LEPR, LIFR, OSMR, and IL31RA), chemokines (e.g., CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, and FAM19A5).

In some embodiments, the proinflammatory cytokine is IL-12, IFN-γ, TNF-α, and/or IL-1β. In some embodiments, the expression of the cytokine is constitutive. In some embodiments, the expression of the cytokine is inducible.

It is contemplated that expression of the proinflammatory cytokine, such as IL-12, enable the engineered immune cells to reprogram and overcome the immunosuppressive TME. Further, the additional expression of the proinflammatory cytokine can enhance tumor antigen presentation, increase tumor-specific cytotoxic T cells activation, and prevent or reduce tumor metastasis.

In some embodiments, the immune cell further expresses a kill switch (or safety module). The kill switch allows the engineered immune cell to be killed or turned off when needed. In some embodiments, the kill switch is a human HSV-TK, a truncated EGFR (tEGFR, e.g., SEQ ID NO:19), or a CD20 protein or fragment. In the case of unacceptable toxicity, the immune cells can be eliminated through administration of a corresponding drug (e.g., ganciclovir) or depleting antibody, (e.g., Cetuximab or Rituximab).

### Polynucleotides and Vectors

The present disclosure also provides polynucleotides or nucleic acid molecules encoding the chimeric receptor, optionally along with other useful components of the engineered immune cell (e.g., proinflammatory cytokine and/or kill switch).

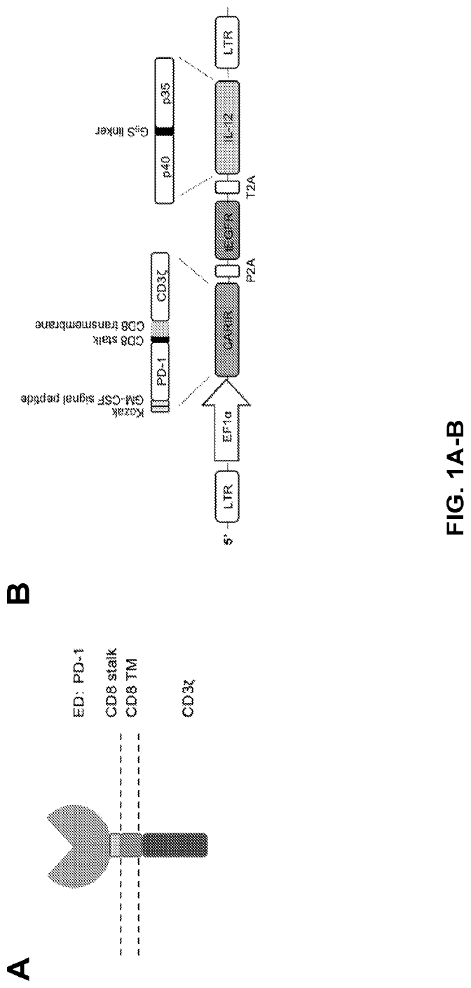

The polynucleotides of the present disclosure may encode chimeric receptor, the proinflammatory cytokine and kill switch on the same polynucleotide molecule (as exemplified in FIG. 1) or on separate polynucleotide molecules.

As illustrated in FIG. 1, the vector encodes a preprotein that includes a leader peptide from human CSF-2, a chimeric receptor that includes an extracellular domain of PD1, a CD8 hinge region, a CD8 transmembrane domain, a CD40 costimulatory domain, the CDξ activation domain, a protease digestion site P2A, a kill switch (truncated EGFR (tEGFR)), a protease digestion site T2A, and an IL-12 (including p40 and p35, connected through a short G₆S linker (SEQ ID NO:15) linker) as the additional proinflammatory cytokine. Upon protease treatments, the preprotein can be split into the chimeric receptor, the tEGFR kill switch, and the IL-12 cytokine.

The polynucleotides encoding desired proteins may be readily prepared, isolated and sequenced using conventional procedures (e.g., by using oligonucleotide probes that are capable of binding specifically to genes encoding the receptor).

Additionally, standard techniques known to those of skill in the art can be used to introduce mutations in the nucleotide sequence encoding a chimeric receptor of the present disclosure, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference chimeric receptor.

The polynucleotides and vectors of the present disclosure can be introduced to a target immune cell with techniques known in the art.

### Cancer Treatment

As described herein, the engineered immune cells of the present disclosure can be used in certain treatment methods. Accordingly, one embodiment of the present disclosure is directed to immune cell-based therapies which involve administering the immune cells of the disclosure to a patient such as an animal, a mammal, and a human for treating one or more of the disorders or conditions described herein.

In some embodiments, the cancer being treated expresses a ligand corresponding to the extracellular targeting domain of the chimeric receptor. Therefore, in some embodiments, cancer cells express PD-L1 and are induced to express PD-L1.

Various cancers can be suitably treated by the instant technology. Non-limiting examples include triple negative breast cancer (TNBC), small cell lung cancer (SCLC), non-small lung cancer (NSCLC), melanoma, glioblastoma, prostate cancer, neuroblastoma, pancreatic ductal carcinoma, urothelial carcinoma, Merkel cell carcinoma, renal cell carcinoma (RCC), Hodgkin lymphoma (cHL), head and neck squamous cell cancer (HNSCC), gastric cancer, cervical cancer, microsatellite instability-high (MSI-H) or mismatch repair deficient (dMMR) metastatic colorectal cancer, and cutaneous squamous cell carcinoma (CSCC).

Some of these cancers, such as NSCLC and SCLC, are currently being treated with anti-PD-L1 antibodies. The instant technology, however, provides cells capable of penetrating the tumors, in particular metastatic tumors and thus can result in improved efficacy. Some of these cancers are currently being tested with the engineered cells of the instant disclosure, with positive results contemplated.

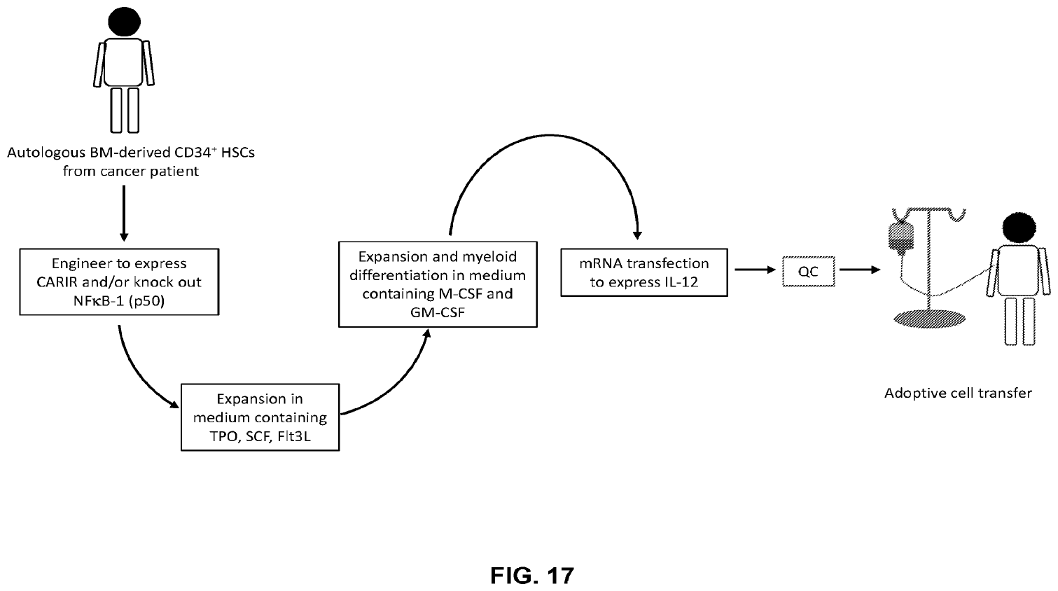

An example adoptive engineered-myeloid cell therapy for treating cancer is illustrated in FIG. 17. At a first step, autologous HSCs are acquired from a cancer patient. Within the HSCs, the p50 gene is inactivated and a CARIR construct is introduced. Then, the transduced HSCs are optionally expanded and differentiated into immature myeloid cells (IMC) or other types of myeloid cells or immune cells, which are transferred back to the patient for treatment.

### Combination Therapies

In a further embodiment, the compositions of the disclosure are administered in combination with a different antineoplastic agent. Any of these agents known in the art may be administered in the compositions of the current disclosure.

In one embodiment, compositions of the disclosure are administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, fludarabine, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In one embodiment, compositions of the disclosure are administered in combination with a checkpoint inhibitor, such as anti-PD-1/PD-L1 or anti-CTLA4 antibodies. In one embodiment, compositions of the disclosure are administered in combination with another cell therapy agent, such as TILs, CAR-T, CAR-NK, CAR-γδT, T-cell antigen coupler (TAC)-T.

In additional embodiments, the compositions of the disclosure are administered in combination with other therapeutic or prophylactic regimens, such as, for example, radiation therapy.

Combination therapies are also provided, which includes the use of one or more of the immune cells of the present disclosure along with a second anticancer (chemotherapeutic) agent. Chemotherapeutic agents may be categorized by their mechanism of action into, for example, anti-metabolites/anti-cancer; purine analogs, folate antagonists, and related inhibitors, antiproliferative/antimitotic agents, DNA damaging agents, antibiotics, enzymes such as L-asparaginase which systemically metabolizes L-asparagine and deprives cells which do not have the capacity to synthesize their own asparagine, antiplatelet agents, antiproliferative/antimitotic alkylating agents, antiproliferative/antimitotic antimetabolites, platinum coordination complexes, hormones, hormone analogs, anticoagulants, fibrinolytic agents, antimigratory agents, antisecretory agents, immunosuppressives, angiotensin receptor blockers, nitric oxide donors, cell cycle inhibitors and differentiation inducers, topoisomerase inhibitors, growth factor signal transduction kinase inhibitors, without limitation.

Additional examples include alkylating agents, alkyl sulfonates, aziridines, emylerumines and memylamelamines, acetogenins, nitrogen mustards, nitrosoureas, anti-metabolites, folic acid analogs, purine analogs, pyrimidine analogs, androgens, anti-adrenals, folic acid replinishers, trichothecenes, and taxoids, platinum analogs.

In one embodiment, the compounds and compositions described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKßE, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

For any of the above combination treatments, the engineered immune cell can be administered concurrently or separately from the other anticancer agent. When administered separately, the engineered immune cell can be administered before or after the other anticancer agent.

### EXPERIMENTAL EXAMPLES

### Example 1. Transduction and Expression of PD-1 CARIR in Human Cells

This example tested lentiviral mediated transduction to and expression in human monocytic THP-1 cells of a Chimeric CAR-like Immune Receptor (CARIR).

A lentiviral vector was constructed to encode the CARIR protein, whose structure is illustrated in FIG. 1. The CARIR included an extracellular domain (ED), a CD8 hinge region, a CD8 transmembrane domain, and the CD35 activation domain. The ED was derived from PD-1. In addition, the lentiviral vector included a protease digestion site P2A, and a kill switch (truncated EGFR (tEGFR)). Upon protease treatments, the expressed preprotein can be split into the chimeric receptor and the tEGFR kill switch.

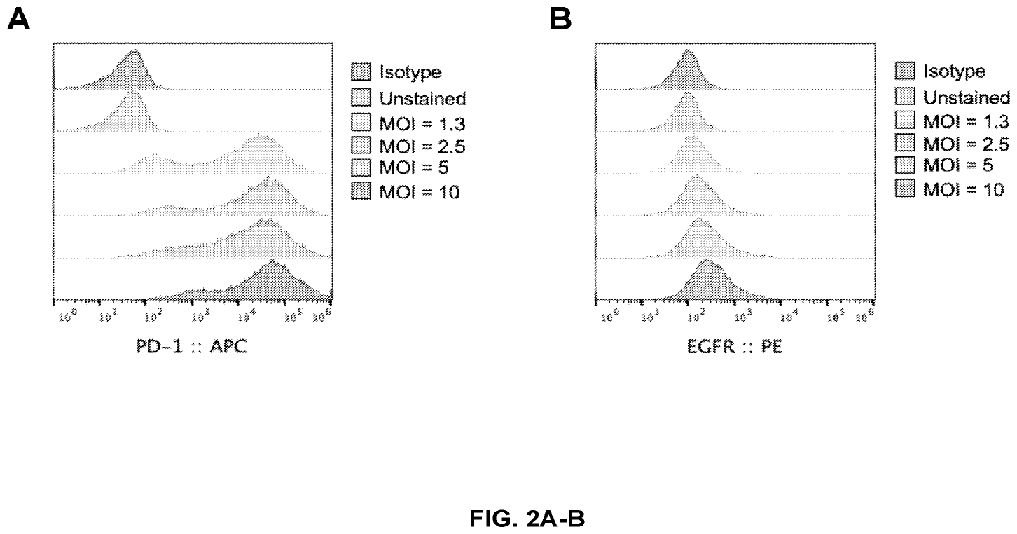

THP-1 cells were transduced with lentiviral vector encoding PD-1 CARIR at certain multiplicity of infection (MOI), including 1.3, 2.5, 5, and 10. The cells were gated on live and singlets. The expression of the CARIR was measured by flow cytometry through detecting surface expression of PD-1. Offset histograms confirmed CARIR expression (FIG. 2A) and the expression of truncated EGFR (FIG. 2B) which serve as a safety kill switch.

### Example 2. CARIR Bound to PD-L1 and Upregulated CD86 and CD80

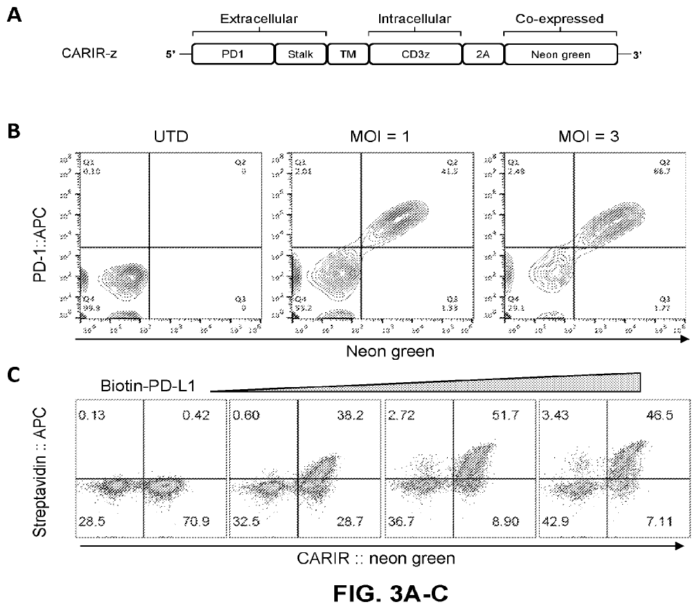

A CARIR constructed, termed CARIR-z (including a CD3ζ activation domain, but not other activation domains), was subjected to binding tests. In addition to CARIR-z, the construct further included a Neon green marker (FIG. 3A). Its expression in the transduced human monocytic THP-1 cells was measured by flow cytometry (FIG. 3B). The transduced THP-1 cells were then incubated with biotinylated human PD-L1 protein. As shown in FIG. 3C, the THP-1 cells bound to the biotinylated human PD-L1.

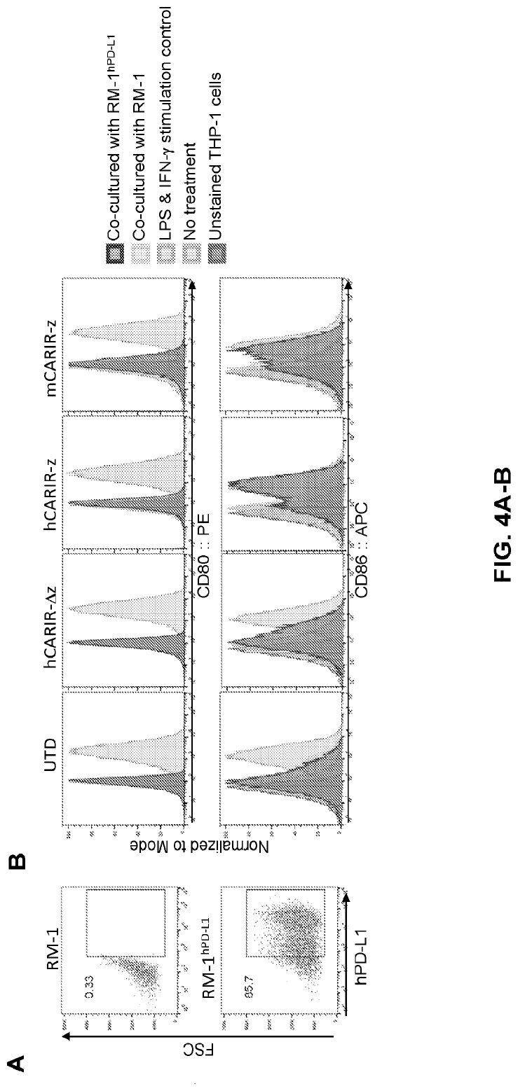

It was then tested whether such binding triggers the proper biological signaling. The CARIR-Δz (with human CARIR that lacks intracellular CD3 zeta chain) and CARIR-Z constructs were used in this assay, along with a mouse counterpart, mCARIR-z which contained the extracellular domain of the mouse PD-1 protein. RM-1 cells transduced with the human PD-L1 protein (RM-1^{hPD-L1}) were confirmed to express PD-L1 (FIG. 4A). The THP-1 cells transduced with the CARIR constructs were then stimulated with 1 ng/ml PMA for 24 hours, followed by the indicated co-culture treatment for 3 days. When the CARIR-expressing THP-1 cells were incubated with the RM-1^{hPD-L1} cells, upregulation of CD86 and, to a much lesser degree, CD80, was observed (FIG. 4B).

### Example 3. Biological Functions of CARIR-Expressing Cells

This example shows that CARIR expression in THP-1 macrophages potentiated pro-inflammatory cytokines production in response to LPS/IFN-γ stimulation.

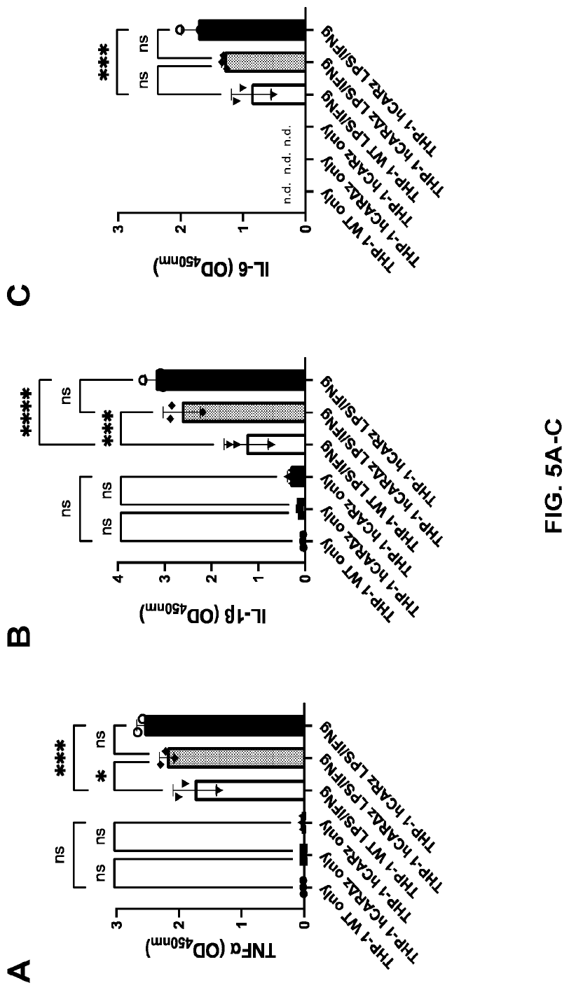

The effector THP-1 cells were pre-treated with 1 ng/ml PMA for 24 hours, and then stimulated with or without LPS+IFN-γ (20 ng/ml for each) for 3 days. The concentration of IL-6 (FIG. 5A), IL-1β (FIG. 5B), and TNF-α (FIG. 5C) cytokines in the culture supernatant was measured by ELISA assay. The experiment was performed in triplicate. The OD₄₅₀ₙₘ value shown was the absorbance (OD) at 450 nm wavelength after the OD value at the reference wavelength of 540 nm was subtracted. The results show that CARIR expression in THP-1 macrophages potentiated pro-inflammatory cytokines production in response to LPS/IFN-γ stimulation.

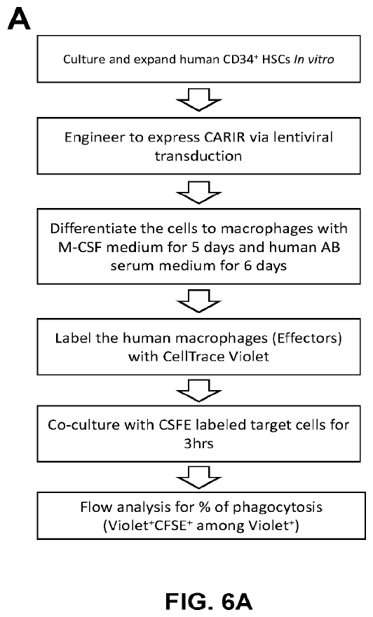

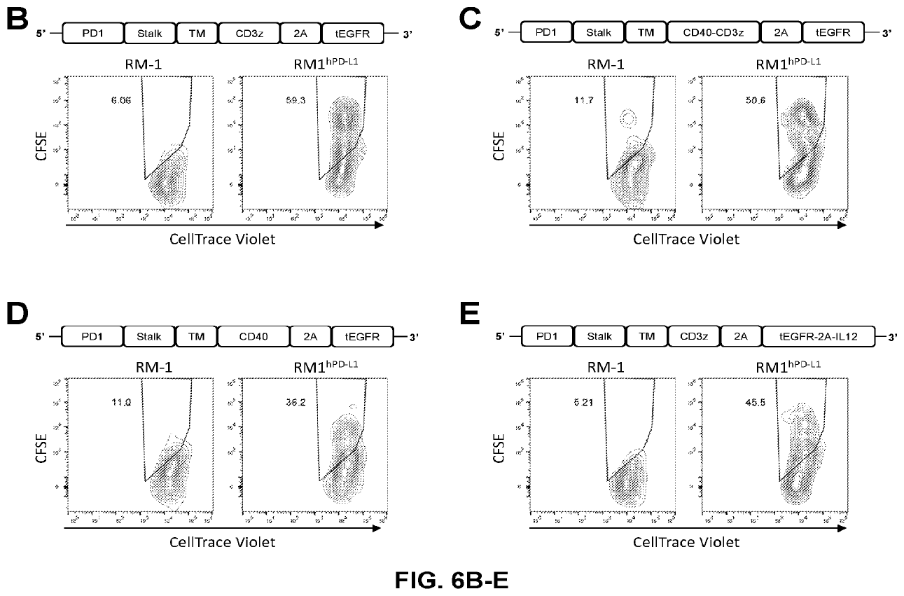

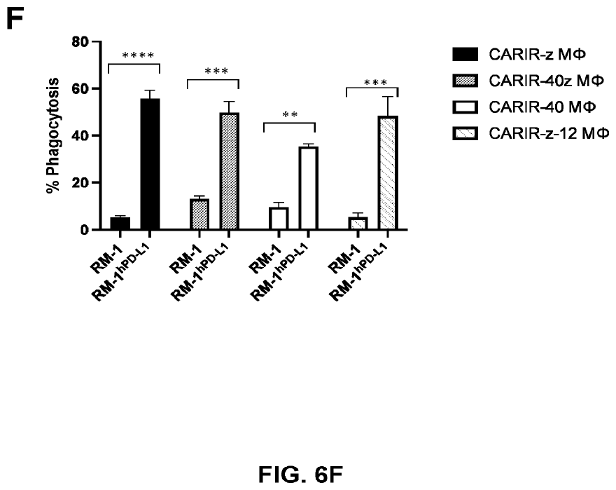

Human CD34⁺ hematopoietic stem cells (HSCs) were engineered to express CARIR through lentiviral transduction, and then differentiated into macrophages (MΦ). For target cells, both wild type RM-1 cells and RM-1^{hPD-L1} cells were used. The later line was established by overexpressing human PD-L1 through lentiviral vector-mediated transduction (see illustration of the experimental procedure in FIG. 6A). The CARIR constructs tested included human CARIR-z (with the intracellular CD3 zeta chain), CARIR-40z (with both the intracellular CD3 zeta chain and CD40 costimulatory domain, and CARIR-z-12 (CARIR-z co-expressed with IL-12). As shown in FIG. 6A-F, CD35 signaling domain is sufficient for CARIR functionality.

In addition, a phagocytosis assay was utilized to evaluate the functionality of CARIR. The CellTrace Violet labeled effector THP-1, CARIR-Δz THP-1 (lacking CD3ζ signaling domain in the CARIR), or CARIR-z THP-1 cells were pretreated with 1 ng/ml PMA for 24 hours. The effector cells were then co-cultured for 4 hours with CFSE labeled RM-1 or RM-1^{hPD-L1} (RM-1 cells that engineered to overexpress human PD-L1) target cells at the effector to target ratio of 5:1, in the presence or absence of 2 μM cytochalasin D (cyto), 10 μg/ml pembrolizumab biosimilar anti-PD-1 antibody (aPD1), or human IgG4 isotype control (iso). The % phagocytosis was assessed by flow cytometry.

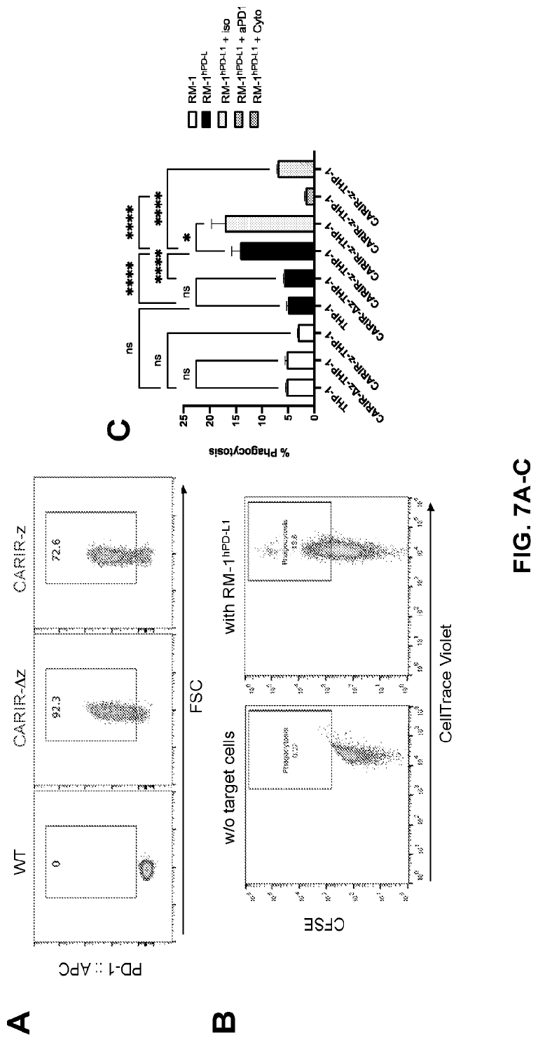

FIG. 7A shows CARIR transduction efficiency in THP-1 cells as evaluated by flow staining for PD-1. The cells were gated on live, singlets. FIG. 7B shows example flow dot plots showing the % phagocytosis by CARIR-z-THP-1 cells in the presence or absence of RM-1^{hPD-L1} target cells. FIG. 7C presents summary comparison charts. CARIR expression in human monocytic THP-1 cells significantly increased phagocytosis on RM-1^{hPD-L1} target cells.

### Example 4. p50-Knockout HSCs

This example prepared hematopoietic stem cells (HSCs) in which the NFκB-1 (p50) gene was knocked out through the CRISPR/Cas9 approach.

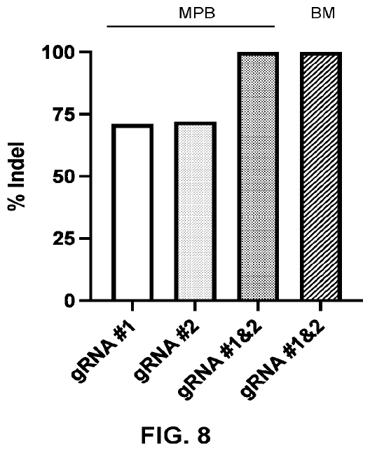

Human mobilized peripheral blood (MPB) or bone marrow (BM)-derived CD34⁺ hematopoietic stem cells (HSCs) were electroporated with ribonucleoprotein (RNP) complex containing recombinant Cas9 protein as well as guide RNA #1 (gRNA #1, target: TACCCGACCACCATGTCCTT, SEQ ID NO:20) and/or guide RNA #2 (gRNA #2, target: ATATAGATCTGCAACTATGT, SEQ ID NO:21). The % indel among the NFκB-1 (p50) gene was measured by TIDE analysis 6 days later, and the results are shown in FIG. 8.

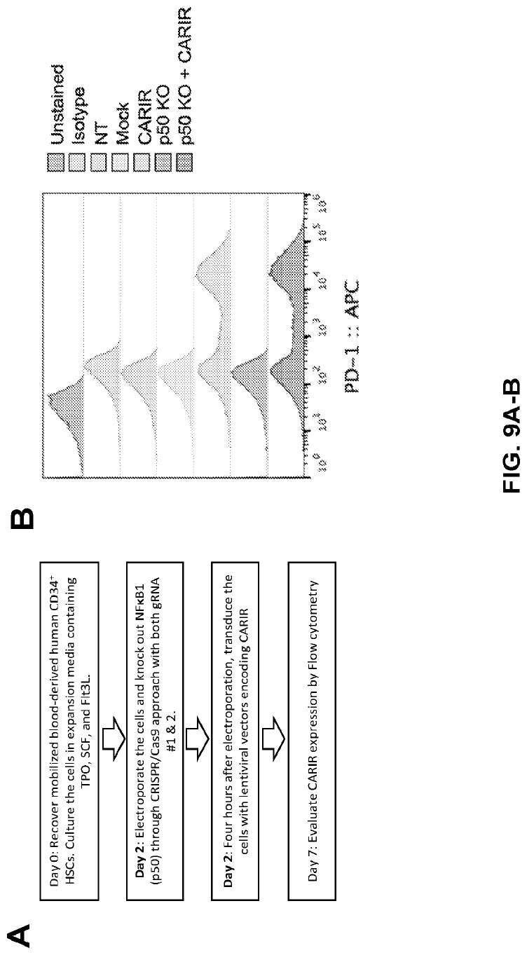

The p50-knockout HSCs were then transduced with, on the same day, constructs encoding the PD-1 CARIR. The procedure is illustrated in FIG. 9A. Offset histograms show CARIR (PD-1) expression on HSCs with or without NF-kB1 (p50) knock out (FIG. 9B).

### Example 5. Expansion and Differentiation of Transduced Cells

This example measured expansion and differentiation of cells transduced with CARIR under different conditions.

The transduced human CD34⁺ hematopoietic stem cells were expanded in vitro for a week in culture medium containing TPO, SCF, Flt3L, and UM171. The number of live cells were counted at the indicated time points. Average numbers of the cells at a few time points are shown in FIG. 10A-B.

MPB- or (BM)-derived human CD34⁺ HSCs were plated in ultralow attachment plates in myeloid differentiation medium containing M-CSF and GM-CSF. The cells were cultured in the incubator that either maintain normoxia (20% O₂) or hypoxia (1% O₂) as indicated. On day 4, 7, and 10, the cells were analyzed by flow cytometry for cell surface markers CD11b and CD34. FIG. 11A-D shows the percentage of CD11b⁺ (A and C), and CD34⁺ (B and D) myeloid cells differentiated under normoxia (A and B) or hypoxia conditions (C and D) over the time course. More myeloid cells resulted from differentiation of BM-versus MPB-derived human CD34⁺ HSCs over a time course of 10 days.

### Example 6. Phagocytosis of Tumor Cells by CARIR-Expressing Monocytic Cells

This example measured phagocytosis of tumor cells by CARIR-expressing monocytic cells.

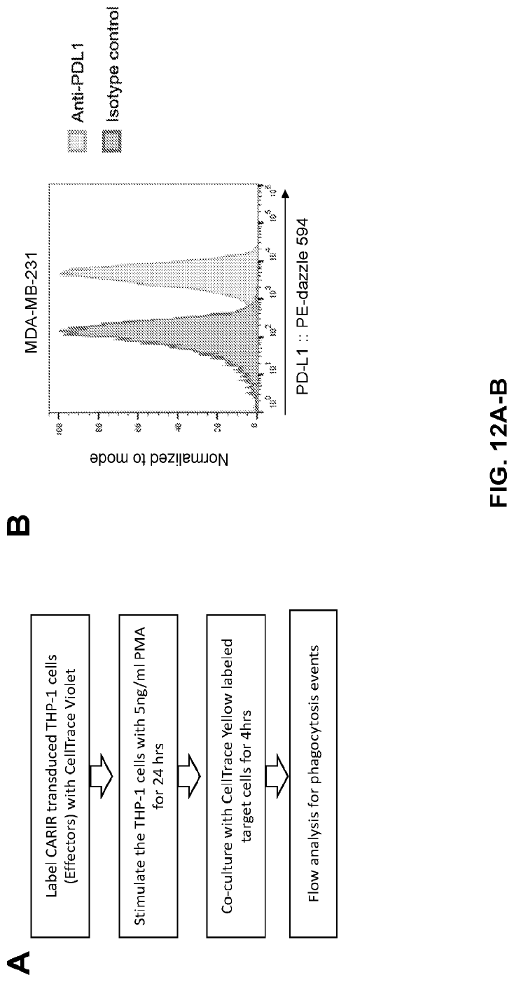

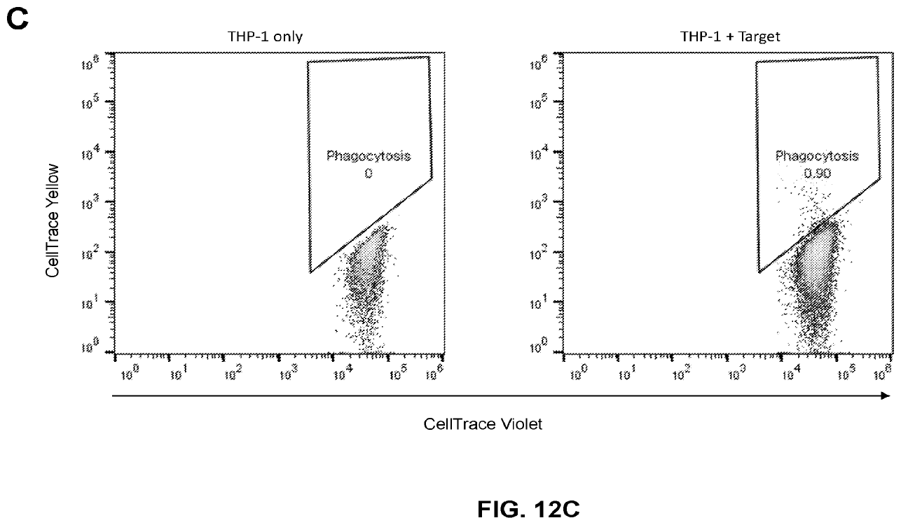

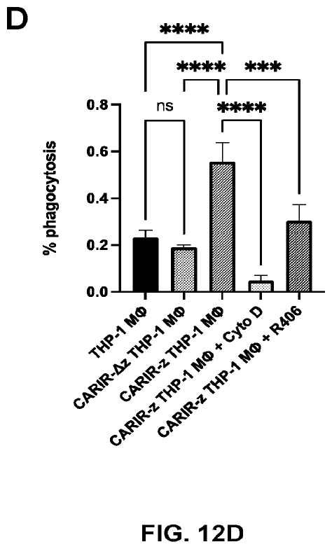

The phagocytosis assay was performed with human triple negative breast cancer cells, and as shown in FIG. 12A-D, CARIR expression in human monocytic THP-1 cells significantly increased their phagocytosis on PD-L1⁺. To serve as the effectors, human monocytic THP-1 cells were engineered to express either CARIR-Δz (a PD-1 CARIR that lacks CD3ζ signaling domain) or CARIR-z through lentiviral transduction, and then differentiated to macrophages by PMA treatment. MDA-MB-231 tumor cells that express PD-L1 were used to serve as the target cells.

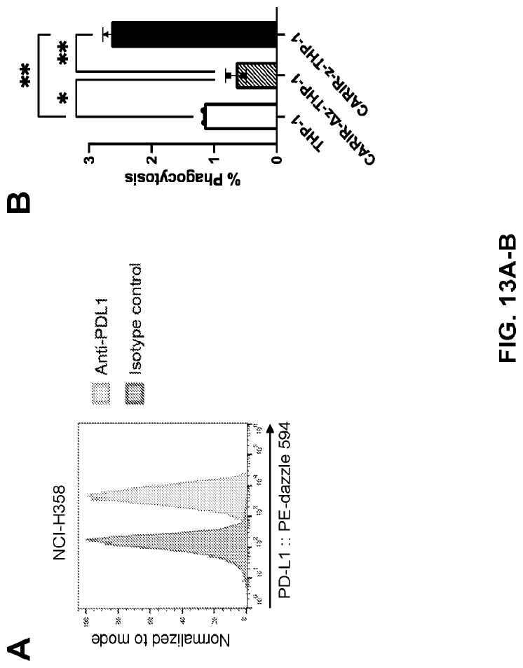

Likewise, as shown in FIG. 13A-B, CARIR expression significantly increased the phagocytosis of human THP-1 macrophages on PD-L1⁺NCI-H358 human lung cancer cells.

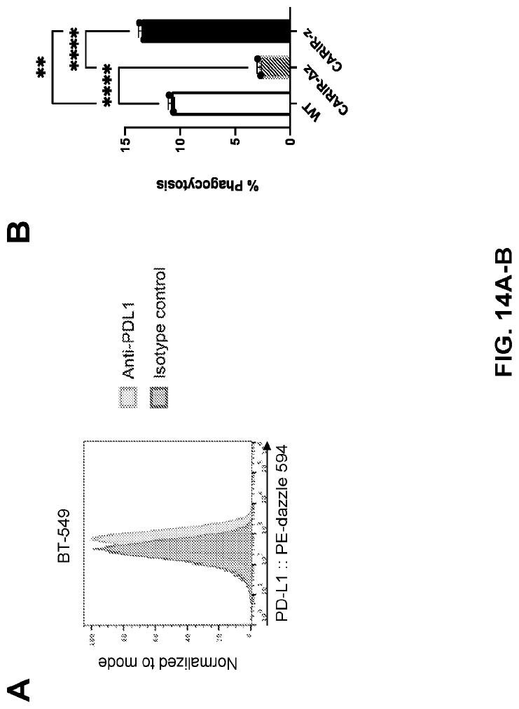

Also, in a similar manner, CARIR expression significantly increased the phagocytosis of human THP-1 macrophages on PD-L1⁺ BT-549 human triple negative breast cancer cells (FIG. 14A-B).

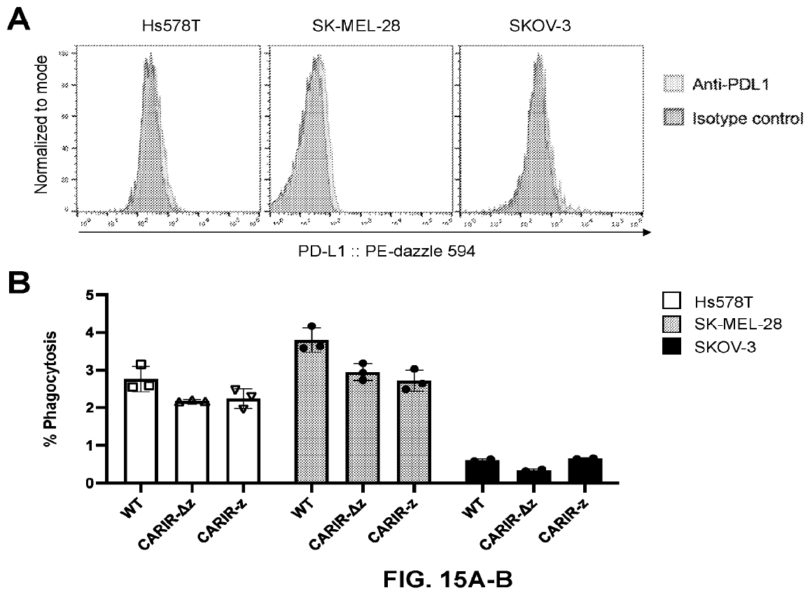

It was then investigated whether the same effect would occur on tumor cells having low or negative PD-L1 expression. To this end, three cell lines were used, including Hs578T, SK-MEL-28 and SKOV-3. As shown in FIG. 15A-B, there was no increase of phagocytosis by CARIR THP-1 macrophages on these PD-L1 low or negative human tumor cells.

### Example 7. In Vivo Testing CARIR-Expressing Myeloid Cells

This example conducted in vivo testing for myeloid cells engineered to express CARIR molecules

A schematic timeline for the in vivo experiment is provided in FIG. 16A. Syngeneic Balb/c mice were subcutaneously implanted with 5×10⁴ 4T1 breast cancer cells on day −7. Starting on day 0, the mice were treated with 3 weekly dose of 10×10⁶ engineered mouse immature myeloid cells expressing murine analog of CARIR (CARIR-IMC) or with NFκB-1 (p50) knocked out (p50^{-/-}-IMC) or PBS as indicated.

The results are shown in FIG. 16B-D, with tumor volume measurements (B) probability of survival (C) and body weight over the course of the experiment (D). Infusion of engineered myeloid cells significantly slowed 4T1 tumor growth and prolonged survival in syngeneic mouse model.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for treating a patient having a tumor cell that expresses programmed death ligand 1 (PD-L1), comprising administering to the patient an immune cell expressing a chimeric receptor comprising, from the N-terminus to the C-terminus, an extracellular domain of programmed cell death-1 (PD-1), a transmembrane domain, a costimulatory domain, and a CD3ξ intracellular domain.

2. The method of claim 1, wherein the tumor cell expresses PD-L1 or is induced to express PD-L1.

3. The method of claim 2, wherein the tumor cell is a cell of a cancer selected from the group consisting of triple negative breast cancer (TNBC), small cell lung cancer (SCLC), non-small lung cancer (NSCLC), melanoma, glioblastoma, prostate cancer, neuroblastoma, pancreatic ductal carcinoma, urothelial carcinoma, Merkel cell carcinoma, renal cell carcinoma (RCC), Hodgkin lymphoma (cHL), head and neck squamous cell cancer (HNSCC), gastric cancer, cervical cancer, microsatellite instability-high (MSI-H) or mismatch repair deficient (dMMR) metastatic colorectal cancer, and cutaneous squamous cell carcinoma (CSCC).

4. The method of claim 2, wherein the tumor cell is a triple negative breast cancer cell.

5. The method of claim 2, wherein the tumor cell is a lung cancer cell.

6. The method of claim 1, wherein the extracellular domain comprises the amino acid sequence of SEQ ID NO:2 or 3.

7. The method of claim 1, wherein the costimulatory domain is a signaling domain of a protein selected from the group consisting of CD28, CD27, OX40, CD40, CD80, CD86, and 4-1BB.

8. The method of claim 1, wherein the immune cell is selected from the group consisting of myeloid cell, natural killer (NK) cell, T cell, tumor infiltrating lymphocyte, and natural killer T (NKT) cell.

9. The method of claim 8, wherein the immune cell is an immature myeloid cell.

10. The method of claim 8, wherein the immune cell is p50 deficient.

11. The method of claim 10, wherein the immune cell does not express an active p50 or has reduced p50 activity.

12. The method of claim 10, wherein the immune cell is a p50 deficient immature myeloid cell.

13. The method of claim 1, wherein the immune cell further comprises an exogenous polynucleotide encoding a proinflammatory cytokine.

14. The method of claim 13, wherein the proinflammatory cytokine is selected from the group consisting of IL-12, IFN-γ, TNF-α, and IL-1β.

15. The method of claim 1, wherein the immune cell further comprises a kill switch.

16. The method of claim 15, wherein the kill switch is selected from the group consisting of HSV-TK, truncated EGFR (tEGFR), and CD20.

17. The method of claim 1, wherein the immune cell was prepared from a cell obtained from the patient.

18. The method of claim 1, wherein the cell was expanded in vitro or ex vivo.

19. The method of claim 16, wherein the cell was expanded under a hypoxic condition.

20. The method of claim 19, wherein the cell was differentiated from a CD34 hematopoietic stem cell obtained from bone marrow.


SEQUENCE LISTING (21 sequences)

SEQ ID NO: 1 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 30 aa, synthetic construct
MSPSNQTDKLAAFPEDRSQPGQDCRFRVTQ

SEQ ID NO: 3 - protein, 101 aa, synthetic construct
MSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 4 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 5 - protein, 17 aa, synthetic construct
MWLQSLLLLGTVACSIS

SEQ ID NO: 6 - protein, 49 aa, synthetic construct
KPTTTPAPRPPTPAPTIASQPLSLRPEACRPAAGGAVHTRGLDFACDIY

SEQ ID NO: 7 - protein, 21 aa, synthetic construct
IWAPLAGTCGVLLLSLVITLY

SEQ ID NO: 8 - protein, 19 aa, synthetic construct
ATNFSLLKQAGDVEENPGP

SEQ ID NO: 9 - protein, 18 aa, synthetic construct
EGRGSLLTCGDVEENPGP

SEQ ID NO: 10 - protein, 62 aa, synthetic construct
KKVAKKPTNKAPHPKQEPQEINFPDDLPGSNTAAPVQETLHGCQPVTQEDGKESRISVQERQ

SEQ ID NO: 11 - DNA, 1599 bp, synthetic construct
  source 1..1599: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagtggaggtggcggcggcggctctcgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 12 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 13 - DNA, 984 bp, synthetic construct
  source 1..984: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagt

SEQ ID NO: 14 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - protein, 7 aa, synthetic construct
GGGGGGS

SEQ ID NO: 16 - DNA, 594 bp, synthetic construct
  source 1..594: mol_type = other DNA
cgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 17 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 18 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 20 - DNA, 20 bp, synthetic construct
  source 1..20: mol_type = other DNA
tacccgaccaccatgtcctt

SEQ ID NO: 21 - DNA, 20 bp, synthetic construct
  source 1..20: mol_type = other DNA
atatagatctgcaactatgt